(19) United States
(12) Patent Application Publication
(10) Pub. No.: US 2005/0069503 A1
(43) Pub. Date: Mar. 31, 2005

(54) Tooth whitening mouthwashes and methods for making and using them

(76) Inventors: Robert K. Larsen, Sandy, UT (US); Calvin D. Ostler, Riverton, UT (US)

Correspondence Address: Parsons Behle & Latimer;Suite 1800, 201 South Main Street, P. O. Box 45898, Salt Lake City, UT, 84111, US

(21) Appl. No.: 10/922,372

(22) Filed: Aug. 20, 2004

Related U.S. Application Data
(63) Continuation-in-part of application No. 10/797,628, filed on Mar. 10, 2004.
(60) Provisional application No. 60/453,467, filed on Mar. 10, 2003.

(51) Int. Cl. A61K 7/20

(52) U.S. Cl. 424/53

(57) ABSTRACT

The addition of the Iodide ion by way of Potassium Iodide to a peroxide such as Hydrogen Peroxide in a basic medium yields Free Radical Oxygen and water; generating large amounts of heat and depleting the Hydrogen Peroxide in a matter of minutes. The Free Radical Oxygen generated in this reaction can be utilized to oxidize organic molecules that produce offending stains on select items, including teeth. Once the Free Radical Oxygen has oxidized the offending molecule the color is lost and the solubility changes allowing the colorless oxidized fragments of the offending molecule to be washed away in the solvent. The Iodide ion catalyzes the reaction allowing for precise control over the speed at which the stain is removed without the need for other expensive, cumbersome energy adding equipment such as lights, lasers, heat sources, etc.

### CROSS REFERENCE TO RELATED APPLICATIONS

This patent application is a continuation-in-part of U.S. patent application Ser. No. 10/797,628 filed on Mar. 10, 2004, which claims benefit to and priority of U.S. Provisional Patent Application Ser. No. 60/453,467 filed on Mar. 10, 2003, and both of the foregoing are hereby incorporated by reference in their entirety.

### BACKGROUND

The disclosure herein relates to tooth whitening mouthwashes and methods for making and using the same.

### SUMMARY

Various tooth whitening mouthwashes and methods for making and using the same are disclosed.

### BRIEF DESCRIPTION OF THE DRAWINGS

FIG. 100 depicts an example bleaching or whitening reaction.

FIG. 200 depicts example hydronium ion acceleration of the liberation of free radical oxygen atoms from the hydrogen peroxide molecule.

FIG. 300 depicts an example of metastable intermediate oxyhydronium ion dissociating into a hydronium ion and the desired free radical oxygen atom.

FIG. 400 depicts by example the addition of a catalyst (such as chemical, heat or light) to the metastable intermediate oxyhydronium ion to drive the dissociation as described in FIG. 300.

FIG. 500 depicts Fenton's reagent.

FIG. 600 depicts an example Bray-Liebhafsky reaction.

FIG. 700 depicts an example of catalytically decomposing hydrogen peroxide.

FIG. 800 depicts example iodide metastable compounds.

FIG. 900 depicts an example formation of bimolecular oxygen.

FIG. 1000 depicts an example of the oxidation of an Iodide ion into Tri-iodide ion by Hydrogen Peroxide.

FIG. 1100 depicts an example disproportionation reaction witnessed at pH levels of 8 and 9.

FIG. 1200 depicts elementary reaction steps pertinent for consideration in formation of oxyhydronium.

FIG. 1300 depicts some of the reaction steps of FIG. 1200 rearranged in sequence.

FIG. 1400 is a plot of data gathered by reacting KI, KCH and KOH in a solution of hydrogen peroxide.

FIG. 1500 is a plot of data gathered by reacting KI, KBr and KOH in a solution of hydrogen peroxide.

FIG. 1600 is a plot of data gathered in acidic and basic reaction experiments.

FIG. 1700 depicts a plot of data gathered in other oxidation reactions.

FIG. 1800 depicts a plot of data gathered in still other oxidation reactions.

FIG. 1900 depicts a plot of results of a KI concentration study.

FIG. 2000 depicts a proposed Sharma reaction sequence.

FIG. 2100 depicts generalized possible reaction types.

FIG. 2200 depicts possible Edelson reaction steps.

FIG. 2300 depicts an Edelson reaction summary.

FIG. 2400 depicts a plot of data gathered during catalyzed decomposition of peroxides.

FIG. 2500 depicts a simple proposed Edelson reaction.

FIG. 2600 depicts Hydrogen Peroxide reacting with the Iodide ion obtained from Potassium Iodide in a basic medium to form metastable intermediaries.

FIG. 2700 depicts a twin chambered containment and mixing vessel for mouthwash.

FIG. 2800 depicts a mixing container for a two-phase mouthwash.

FIG. 2900 depicts use of a mouthwash.

### DETAILED DESCRIPTION

The description herein should be read in conjunction with the appended drawings, and the reference numerals used refer to the drawings. The entirety of the disclosure herein, including the specifics thereof, are intended to be exemplary and not limiting.

It is well established that the free radical oxygen atoms (140) liberated from peroxides such as hydrogen peroxide (130), carbamide peroxide, and salts of peroxides formed from the alkali and alkaline earth metals, readily attack and oxidize organic molecules (160) that comprise the stains in discolored teeth. It is also well established that a release of free radical oxygen atoms from the peroxides can be accelerated by the addition of heat, light and/or chemicals; specifically chemicals that raise the pH of the peroxide environment. A lengthy dissertation of the exact mechanisms is discussed in prior work found in U.S. Pat. No. 6,116,900, “Binary energizer and peroxide delivery system for dental bleaching” which is herein incorporated by reference.

The use of alkali metal and alkaline earth metal salts of the hydroxyl group and alkali metal and alkaline earth metal salts of the carbonate and bicarbonate groups to increase the pH to accelerate the release of free radical oxygen atoms in dental bleaching compositions has been exhaustively explored and reported. The use of alkali metal and alkaline earth metal salts of the hydroxyl group and alkali metal and alkaline earth metal salts of the carbonate and bicarbonate groups to increase the pH and stabilize the gels formed by polyacrylic acid thickeners have likewise been exhaustively explored and reported. However, the hydroxyl group, more specifically the hydronium ion, OH—, has only limited potential to increase the generation of the free radical oxygen. Hydronium ion acceleration of the liberation of free radical oxygen atoms from the hydrogen peroxide molecule proceeds according to the reaction described in FIG. 200. The hydronium ion (220) is produced by dissociation of alkali metal and alkaline earth metal salts mentioned above. When the hydronium ion (220) is mixed with the peroxide (210), water (240) and the metastable intermediate oxyhydronium ion (230) is produced. From here the reaction proceeds according to the equation in FIG. 300. The metastable intermediate oxyhydronium ion (310), when allowed to sit for a time, will dissociate into a hydronium ion (320) and the desired free radical oxygen atom (330). Kinetically, this is the rate-limiting step. One can drive the formation of the metastable intermediate oxyhydronium ion (310) by adding additional hydronium ion, however, without the addition of unwanted energy such as heat, the dissociation of the metastable intermediate oxyhydronium ion (310) to the desired free radical oxygen atom (330) is time dependent. The addition of a catalyst to the metastable intermediate oxyhydronium ion (310) would chemically drive the dissociation as described in FIG. 300. Refer to FIG. 400. The addition of the catalyst (420) to the system greatly reduces the time required for the reaction to continue. By increasing or decreasing the concentration of the catalyst, one can control the overall rate of production of the desired free radical oxygen atom (440).

The first peroxide, hydrogen peroxide, was discovered in 1818. Consequently, its bleaching ability is very well known and chemical literature and history is replete with examples of the catalytic “decomposition” of hydrogen peroxide. (The quotation marks are included around “decomposition” to denote the fact that any specific definition of or for “decomposition” is not necessarily agreed upon. We use the term “decomposition” as it is applied to peroxide to denote the change of peroxides to any number of subspecies.) Nearly as old a discovery as hydrogen peroxide and the most widely known of the peroxide catalysts is Fenton's Reagent. Refer to FIG. 500. Fenton's Reagent is particularly instructive because the Iron ions themselves (510 and 520) react as controllable catalysts only in a very carefully controlled pH environment (530) and when the Hydrogen Peroxide (540) is added slowly. If the iron ions (510 and 520) are allowed to react at a pH of greater than 5 (550) Iron will precipitate our of the solution (560) and the catalytic decomposition of Hydrogen Peroxide into binary Oxygen will proceed uncontrollably liberating a great amount of heat, creating a dangerous situation for all involved.

Much work has been performed since the discovery of peroxides and Fenton's Reagent, however, the bulk of useful catalytic peroxide chemistry has been discovered in the past 50 years. The overwhelming majority of these reactions involve the catalytic activity of Transition Metals and Transition Metal Oxides. Such chemistry has been commercially used in end-of-pipe treatment of effluent of chemical industries. While catalytic use of the Metals and Metal Oxides are usable for industry they become not unusable but less satisfactory for use by average consumers because of the problems illustrated in the Fenton's Reagent example above. Namely, they generally require precise environmental conditions in order to be controlled and in many situations undesirable precipitants are created such the Iron precipitant described in the Fenton's Reagent example above.

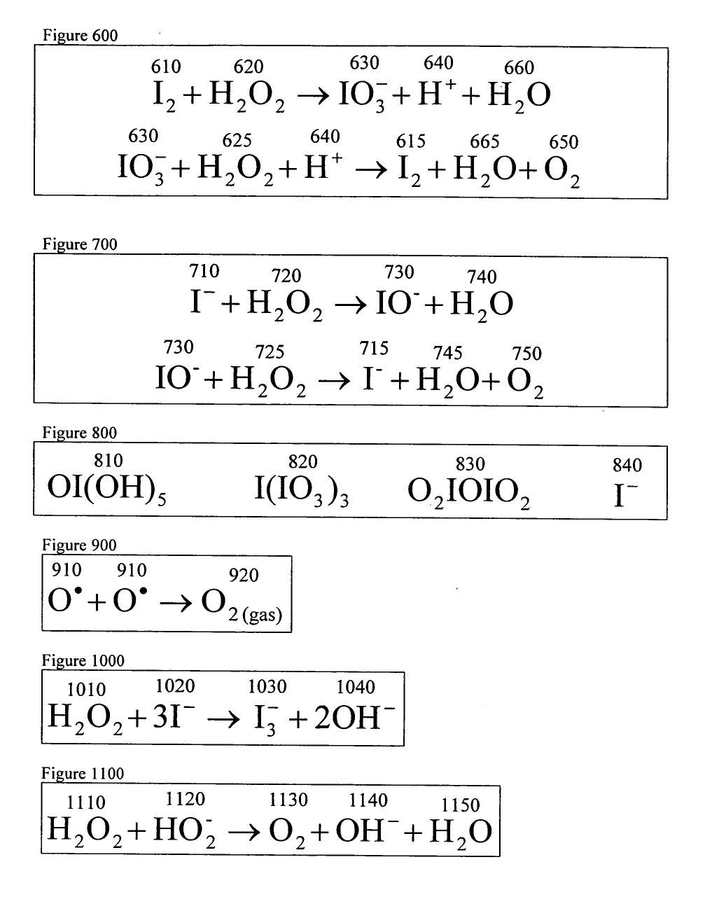

It has long been known that Hydrogen Peroxide is unstable in the presence of Iodide. Iodide is a potential solution to the problems that are inherent in the Metal and Metal Oxide systems. The most commonplace and most understood of the Iodide-Peroxide reactions is the Bray-Liebhafsky Reaction which was reported in 1921. However, the Bray-Liebhafsky Reaction is described as a decomposition reaction not a free radical generating reaction. Refer to FIG. 600. Iodine (610) is added to Hydrogen Peroxide (620). This reaction yields the Iodate ion (630), a proton (640) and water (660). The lodate ion (630) then begins a second reaction with additional Hydrogen Peroxide (625), however, it cannot proceed without a proton (640) the subsequent reaction yields Iodine (615), more water (665) and bimolecular Oxygen (650) which is evolved as a gas. In this reaction Iodine rocks back and forth from Iodine to lodate, hence the famous “Oscillatory” nature of the famous reaction. In itself this is a problem for consumer type whitening because Iodine is purple and creates yellow to brown to purple stains plus, as a purely decomposing reaction, the Bray-Liebhafsky Reaction produces no free radicals. While oxidation clearly happens with bimolecular oxygen it is geologically slow relative to free radical oxidation. Therefore, on its face, acidic-catalytic “decomposition” of peroxides by Iodide would be useless to consumer whitening or bleaching. Therefore, Iodide has been dismissed as a useless component of any type of peroxide based whitening or bleaching system.

We recognized, as do many, that peroxides are less stable in a basic medium. We further recognized that Iodide will not be reduced and precipitate out of a basic medium. We conducted many laboratory experiments to determine feasibility of such a basic system in which an ionic catalytic reaction could occur with peroxides. We chose Iodide for the reasons explained above and Hydrogen Peroxide for its ease of use, but have confirmed the results with other peroxides. The first experiment performed was to place Hydrogen Peroxide in a basic solution to which Potassium Iodide was added. Immediately small bubbles formed in the system. As time passed the rate of the reaction increased to a very rapid liberation of bimolecular Oxygen and heat. The first speculation was that we were catalytically decomposing the Hydrogen Peroxide by a well understood and agreed upon mechanism. Refer to FIG. 700. Iodide (710) is added to Hydrogen Peroxide (720). The reaction produces an Iodide-Oxygen anion (730) and water (740). The Iodide-Oxygen anion (730) then proceeds in another reaction with more Hydrogen Peroxide (725). That reaction produces another Iodide ion (715) which will be used in the first reaction, hence the reaction is catalytic, more water (745), and bimolecular Oxygen (750) by way of a gas that is seen bubbling out of the solution. As stated, this “Decomposition” reaction mechanism is well known and can even be substituted in the Bray-Liebhafsky Reaction in which the lodate ion of the Bray reaction is replaced with the Iodide ion in this reaction. In other words, as they are understood and agreed upon, it is possible that both or either mechanism is/are at work regardless of the pH. However, we confirmed experimentally, with Starch Indicator, that there is elemental Iodine (Iodine is a zero oxidation state) present in the acidic medium reaction and no elemental Iodine present in the basic medium reaction, suggesting that the two mechanisms were very different. Upon further investigation we discovered that Iodide forms several metastable compounds, in different oxidation states, in basic aqueous solutions. Refer to FIG. 800. In first Iodine metastable complex (810) Iodine is in the plus 7 oxidation state. In the second metastable Iodine complex (820) four Iodine ions are present, 2 are in the plus 7 oxidation state and one is in the plus 5 oxidation state and one is in the plus 1 oxidation state. In the third metastable Iodine complex there are Iodine ions both of which are in the plus 5 oxidation state. It is therefore possible that all of the allowable positive oxidation states for Iodine, +1, +5, +7, are present in the solution. If all of the allowable positive oxidation states for Iodine are present in the solution a free radical mechanism is possible.

It was reasoned that if the reaction were simply a decomposition reaction, other Halide ions would produce the same or similar decomposition rates and the same or similar amounts of heat and bimolecular Oxygen. Experiments were conducted using Potassium Chloride and Potassium Bromide in basic solutions of Hydrogen Peroxide. No reactions were noted over extended amounts of time. We summarized that the reaction was not decomposing Hydrogen Peroxide as discussed above and illustrated in FIG. 700 but was generating free radicals as discussed above and illustrated in FIG. 400. We further speculated that the bimolecular Oxygen being generated in this reaction was not being generated by the direct decomposition of Hydrogen Peroxide as illustrated in FIG. 700 but (Refer to FIG. 900) was being generated by way of two free radical Oxygen atoms (910) combining to form bimolecular Oxygen (920).

It was reasoned that if an abundance of free radical Oxygen was being formed the free radicals would bleach or whiten very rapidly. A laboratory experiment was set up to confirm the presence of free radicals.

A potassium hydroxide and potassium iodide solution was prepared by adding 0.066 grams of potassium hydroxide and 0.90 grams of potassium iodide to 100 milliliters of distilled water.

20 milliliters of 20% hydrogen peroxide aqueous solution was placed in a beaker.

20 milliliters of the potassium hydroxide and potassium iodide solution was added to the 20% hydrogen peroxide solution. A stained cow's tooth was added to the mixture. After 10 minutes of exposure to the solution the stained cow's tooth has been whitened by 16 shades.

A control tooth placed in an aqueous hydrogen peroxide solution of exactly the same concentration but without the potassium hydroxide and potassium iodide did not perceptibly lighten or whiten in the same amount of time.

The free radical nature of the reaction being confirmed by laboratory work, an extensive search of the chemical literature with respect to the Iodide catalyzation and Iodide decomposition of peroxides was conducted and the following references were found:

Mechanisms of hydrogen peroxide decomposition in soils. Petigara, Bhakti R.; Blough, Neil V.; Mignerey, Alice C. Department of Chemistry and Biochemistry, University of Maryland, College Park, Md., USA. Environmental Science and Technology (2002), 36(4), 639-645.

Mechanism of the decomposition of hydrogen peroxide under alkaline conditions. Yokoyama, Tomoya. Department of Wood and Paper Science, North Carolina State University, Japan. Cellulose Communications (2001), 8(1), 16-20.

Kinetics and mechanisms of decomposition reaction of hydrogen peroxide in presence of metal complexes. Salem, Ibrahim A.; El-Maazawi, Mohamed; Zaki, Ahmed B. Department of Chemistry, United Arab Emirates University, Al-Ain, United Arab Emirates. International Journal of Chemical Kinetics (2000), 32(11), 643-666.

Systematic design of chemical oscillators. 44. Kinetics and mechanism of hydrogen peroxide decomposition catalyzed by copper (2+) in alkaline solution. Luo, Yin; Kustin, Kenneth; Epstein, Irving R. Dep. Chem., Brandeis Univ., Waltham, Mass., USA. Inorganic Chemistry (1988), 27(14), 2489-96. CODEN: INOCAJ ISSN: 0020-1669.

“Complex” versus “free radical” mechanism for the catalytic decomposition of hydrogen peroxide by ferric ions. Kremer, Mordechai L. Dep. Phys. Chem., Hebrew Univ. Jerusalem, Jerusalem, Israel. International Journal of Chemical Kinetics (1985), 17(12), 1299-314.

Reactions involving hydrogen peroxide, iodine, and iodate ion. 7. The smooth catalytic decomposition of hydrogen peroxide, mainly at 50□C. Liebhafsky, Herman A.; Furuichi, Ryusaburo; Roe, Glenn M. Dep. Chem., Texas A and M Univ., College Station, Tex., USA. Journal of the American Chemical Society (1981), 103(1), 51-6.

Oscillations in chemical systems. 13. A detailed molecular mechanism for the Bray-Liebhafsky reaction of iodate and hydrogen peroxide. Sharma, Kumud R.; Noyes, Richard M. Dep. Chem., Univ. Oregon, Eugene, Oreg., USA. Journal of the American Chemical Society (1976), 98(15), 4345-61.

Formation spectra and some chemical properties of oxyiodine radicals in aqueous solutions. Tendler, Y.; Faraggi, M. Nucl. Res. Cent.-Negev, At. Energy Comm., Beer-Sheva, Israel. Journal of Chemical Physics (1973), 58(3), 848-53.

Effect of pH on the system I-/I3-/H2O2. Application to iodine hydrolysis. Kessi-Rabia, M.; Gardes-Albert, M.; Julien, R.; Ferradini, C. Institut Chimie, Universite des Sciences et de la Technologie, Algiers, Algeria. Journal de Chimie Physique et de Physico-Chimie Biologique (1995), 92(5), 1104-23.

Reactions of iodine intermediates in iodate-hydrogen peroxide oscillators. Furrow, Stanley. Pennsylvania State Univ., Reading, Pa., USA. Journal of Physical Chemistry (1987), 91(8), 2129-35.

Studies on singlet oxygen in aqueous solution. Part 4. The ‘spontaneous’ and catalyzed decomposition of hydrogen peroxide. Evans, Dennis F.; Upton, Mark W. Inorg. Chem. Lab., Imp. Coll., London, UK. Journal of the Chemical Society, Dalton Transactions: Inorganic Chemistry (1972-1999) (1985), (12), 2525-9.

Spectrophotometric determination of inorganic iodine compounds and hydrogen peroxide in neutral and slightly alkaline solutions. Habersbergerova, A. Nucl. Res. Inst., Rez, Czech. Radiochemical and Radioanalytical Letters (1977), 28(5-6), 439-43.

Solvation and salt effects in the reaction of hydrogen peroxide with iodide ion at high iodide concentrations. Surfleet, B.; Wyatt, Peter A. H. Univ. Sheffield, Sheffield, UK. Journal of the Chemical Society [Section] A: Inorganic, Physical, Theoretical (1967), (10), 1564-6.

Interaction of hydrogen peroxide with potassium iodide, and its use in the estimation of chromium. Rupp, E.; Hamann, G.; Muller, R. Arch. Pharm. (1934), 272 57-60.

Radical and molecular yields in the [ ]-radiolysis of water. II. The potassium iodide-nitrous oxide system in the pH range 0-14. Buxton, O. V.; Dainton, F. S. Univ. Leeds, UK. Proc. Roy. Soc. (London) Ser. A (1965), 287(1411), 427-43.

Rates of reaction of the hydroxyl radical. Thomas, J. K. Argonne Natl. Lab., Argonne, Ill., Trans. Faraday Soc. (1965), 61(508), 702-7.

The action of □-rays of 60Co on neutral or alkaline solutions of potassium iodide in the presence of air. Jove, Jose; Pucheault, Jacques. Inst. Radium, Paris, Journal de chimie physique et de physico-chimie biologique. (1964), 61(5), 711-16.

Radiation chemistry studies of aqueous iodine-iodide solutions. Senvar, Cemil B. Commun. Fac. Sci. Univ. Ankara Ser. B (1962), 10 1-6.

Reactions involving hydrogen peroxide, iodine, and iodate ion. V. Introduction to the oscillatory decomposition of hydrogen peroxide. Liebhafsky, Herman A.; Wu, Lawrence S. Dep. Chem., Texas A and M Univ., College Station, Tex., USA. Journal of the American Chemical Society (1974), 96(23), 7180-7.

Catalytic decomposition of hydrogen peroxide in alkaline solutions. Venkatachalapathy, Rajeev; Davila, Guadalupe P.; Prakash, Jai. Center for Electrochemical Science and Engineering, Department of Chemical and Environmental Engineering, Illinois Institute of Technology, Chicago, Ill., USA. Electrochemistry Communications (1999), 1(12), 614-617.

Decomposition of H2O2 over manganese-chromium oxide catalyst in aqueous and alkaline solutions. Selim, M. M.; El-Aiashi, M. K.; Mazhar, H. S.; Kamal, S. M. Natl. Res. Cent., Cairo, Egypt. Materials Letters (1996), 28(4-6), 417-421. CODEN: MLETDJ ISSN: 0167-577×.

Decomposition of alkaline solutions of hydrogen peroxide with inorganic salt additions. Tumanova, T. A.; D'yachenko, Yu. I.; Puzyrev, S. S. Leningr. Lesotekh. Inst., Leningrad, USSR. Izvestiya Vysshikh Uchebnykh Zavedenii, Khimiya i Khimicheskaya Tekhnologiya (1988), 31(4), 21-5.

Catalytical activity of manganese dioxide for hydrogen peroxide decomposition in alkaline solutions. Zalyoksnis, Y.; Tryk, D.; Yeager, E. Case Lab. Electrochem. Sci., Case West. Reserve Univ., Cleveland, Ohio, USA. Battery Material Symposium, [Proceedings] (1985), 2nd 467-76.

Alkali-induced generation of superoxide and hydroxyl radicals from aqueous hydrogen peroxide solution. Csanyi, L. J.; Nagy, L.; Galbacs, Z. M.; Horvath, I. Inst. Inorg. Anal. Chem., A. Jozsef Univ., Szeged, Hung. Zeitschrift fuer Physikalische Chemie (Munchen, Germany) (1983), 138(1), 107-16.

Kinetics of the decomposition of hydrogen peroxide in alkaline solutions. Spalek, Otomar; Balej, Jan; Paseka, Ivo. Inst. Inorg. Chem., Czechoslovak Acad. Sci., Prague, Czech. Journal of the Chemical Society, Faraday Transactions 1: Physical Chemistry in Condensed Phases (1982), 78(8), 2349-59.

Generation of superoxide radicals in alkaline solutions of hydrogen peroxide and the effect of superoxide dismutase in this system. Csanyi, Laszlo J.; Galbacs, Zoltan M.; Horvath, Laszlo. Inst. lnorg. Anal. Chem., A. Jozsef Univ., Szeged, Hung. Inorganica Chimica Acta (1981), 55(1), 1-4.

Decomposition of hydrogen peroxide in dilute alkaline aqueous solutions. Makkonen, Hannu P. Univ. Washington, Seattle, Wash., USA. Avail. Xerox Univ. Microfilms, Ann Arbor, Mich., Order No. 74-29,457. (1974), 92 pp. From: Diss. Abstr. Int. B 1975, 35(7), 3229-30.

Kinetics and mechanism of the spontaneous decompositions of some peroxy acids, hydrogen peroxide, and tert-butyl hydroperoxide. Koubek, E.; Haggett, M. L.; Battaglia, C. J.; lbne-Rasa, Khairat M.; Pyun, H. Y.; Edwards, J. O. Brown Univ., Providence, RI, J. Am. Chem. Soc. (1963), 85(15), 2263-8.

Thermoanalytic investigation of the catalytic decomposition of hydrogen peroxide by palladium solutions, with special regard to fluoride ions. Tamura, M.; Ishizuka, S.; Kono, T.; Uruha,. Kyoto Furitsu Ika Daigaku Zasshi (1957), 62 577-82.

Interaction of poly(vinylpyrrolidinone) and iodine. Cournoyer, Robert F.; Siggia, Sidney. Dep. Chem., Univ. Massachusetts, Amherst, Mass., USA. Journal of Polymer Science, Polymer Chemistry Edition (1974), 12(3), 603-12.

Chemistry of iodine (I) in alkaline solution. Chia, Yuan-Tsan. Univ. of California, Berkeley, U.S. Atomic Energy Comm. (1958),

Halogen oxy compounds. XI. Kinetics of the formation of iodate from hypoiodite for small iodide concentrations. Skrabal, A.; Hohlbaum, R. J. Chem. Soc. (1916), 110(11), 477.

Supersaturation Limit for Homogeneous Nucleation of Oxygen Bubbles in Water at Elevated Pressure: “Super-Henry's Law”. Bowers, Peter G.; Hofstetter, Christine; Letter, Caroline R.; Toomey, Richard T. Department of Chemistry, Simmons College, Boston, Mass., USA. Journal of Physical Chemistry (1995), 99(23), 9632-7.

Detailed Calculations Modeling the Oscillatory Bray-Liebhafsky Reaction. Edelson, David; Noyes, Richarch M. Journal of Physical Chemistry (1979) 83(2), 212-220.

The above references are herein incorporated by reference in their entirety.

A distillation of the above reference is represented in three of the articles that we will discuss at length. The first is: Effect of pH on the system I-/I3-/H2O2. Application to iodine hydrolysis. Kessi-Rabia, M.; et al. This document is hereby incorporated by reference in its entirety.

In this study, Kessi-Rabia ran the reactions at four different pH levels: 4.7, 7, 8, and 9. In order to achieve the desired pH levels appropriate pH buffer systems were employed; this held the pH constant during the course of the reaction. Kessi-Rabia elected to first study the end products of the reactions at the various pH levels. The reaction products measured were bimolecular Oxygen and the Tri-iodide species. Refer to FIG. 1000. The oxidation of the Iodide ion (1020) into The Tri-iodide ion (1030) by Hydrogen Peroxide (1010) is a well known reaction. Note that the reaction produces the Hydroxyl ion (1040). Kessi-Rabia plotted the final concentration of the Tri-iodide ion at the four pH levels and found that a relatively large concentration of Tri-iodide ion existed at the lower pH value of 4.7. However, at a pH of 7 the concentration of the Tri-iodide ion begins to fall off, at a pH value of 8 there is very little of the Tri-iodide ion left, and at a pH level of 9 the Tri-iodide ion is gone. Bimolecular Oxygen, on the other hand, demonstrated nearly an exact inverse relationship to the Tri-iodide; at a pH of 4.7 there was virtually no bimolecular Oxygen, at a pH of 7 the concentration of bimolecular Oxygen begins to rise, at a pH of 8 there is a great deal, relatively, of bimolecular Oxygen, and at the pH level of 9 the bimolecular Oxygen is higher than that obtained at a pH level of 8 but the curve has flattened. The curve of the bimolecular Oxygen is very nearly a mirror image of the curve of the Tri-iodine ion. The curves cross at a pH level of approximately 7.5. Kessi-Rabia states that at the pH level of 4.7 and lower the reaction illustrated in FIG. 1000 and described above dominate the system and dominate to a lesser extent in pH systems of 7 and 8 given the condition that the Iodide (1020) are much greater than the concentrations of Hydrogen Peroxide (1010).

Keesi-Rabia further states that the “disproportionation” reaction witnessed at pH levels of 8 and 9 are described by the reaction in FIG. 1100. The Oxyhydronium ion (1120) [the creation of the Oxyhydronium ion in basic solution is illustrated in FIG. 200 and discussed above] reacts with Hydrogen Peroxide (1110) to form bimolecular Oxygen (1130), a Hydroxyl ion (1140) and water (1150). Of course such an equation fails to account for any effect of Iodine in any oxidation state.

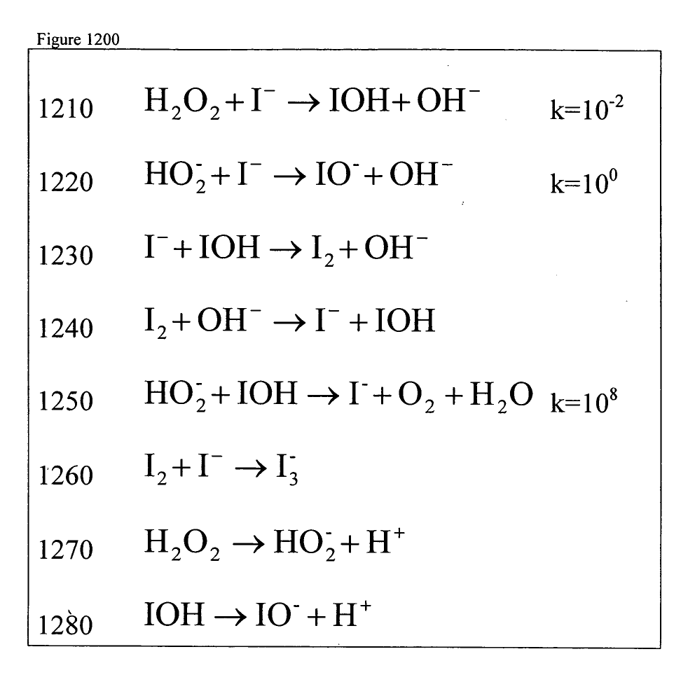

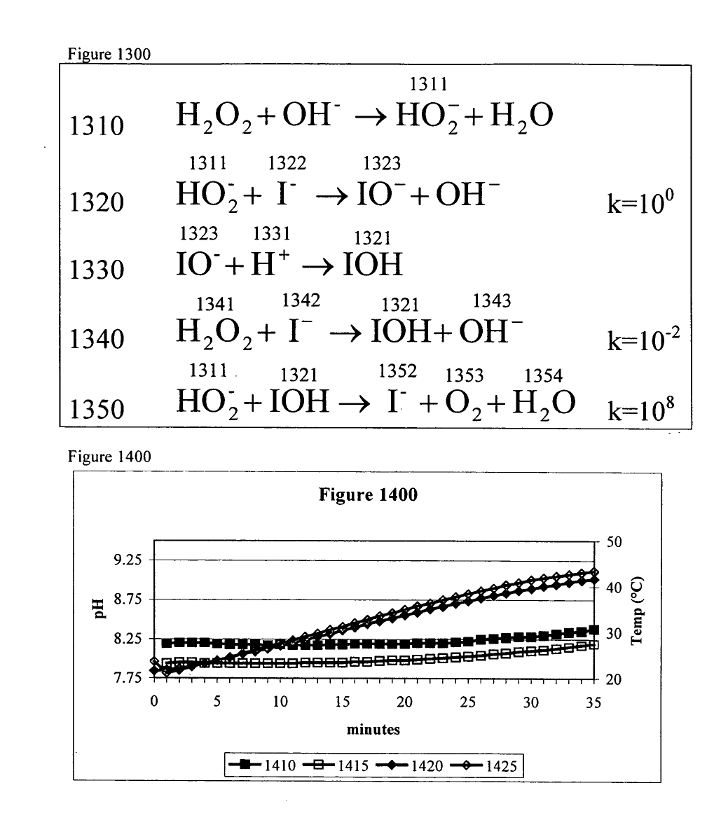

Kessi-Rabia instructs us to consider the elementary steps illustrated in FIG. 1200 to explain the results. At first glance an immediate determination that the elementary steps of 1230, 1240, and 1260 must be discarded because no elemental (zero oxidation state) Iodine exists in the basic solution. Steps 1270 and 1280 assumed equilibria depending on the pH of the system. The step 1270 is predominately shifted to the right in basic solution as illustrated in FIG. 200 and discussed above. The precursor to step 1280 is step 1210. These remaining, suggested, elementary steps are rearranged and presented in FIG. 1300. The steps are arranged in their logical order in terms of requisite reaction sequences. Step 1310 is a basic solution modification of 1270, is also illustrated in FIG. 200 and discussed above. Step 1310 describes the formation of the requisite Oxyhydronium ion, the Oxyhydronium being a reactant in steps 1320 and 1350. The next step is to produce the other reactant required in step 1350, the IOH molecule ((1321). Kessi-Rabia has provided us with two different mechanisms to produce the IOH molecule. First (1320) the Oxyhydronium ion (1311) reacts with the Iodide ion (1322) to produce the 10 anion (1323). The 10 anion (1323) is then added to step 1330 where it reacts with a proton (1331) to form the IOH molecule (1321). It is unclear where the proton (1331) comes from in this reaction as the solution is decidedly basic. The second mechanism proposed by Kessi-Rabia to form the IOH molecule (1321) is illustrated as step 1340. In step 1340, Hydrogen Peroxide (1341) reacts with Iodide (1342) to produce the needed IOH molecule (1321) and a Hydroxyl ion (1343). The K value is very low for this reaction and because the solution is again, decidedly, basic it is reasonable to assume that this reaction would be forced to the left or not proceed at all. Because the only step available in this proposal to form the requisite IOH molecule (1321) actually requires a proton to proceed it is the least likely of two unlikely mechanisms to proceed. We will therefore assume for the sake of this discussion that step 1340 does, in fact, move to the right and produces the IOH molecule (1321). We now proceed to step 1350 where the described “disproportionation” reaction actually takes place. The Oxyhydronium ion produced in step 1310 reacts with the IOH molecule (1321) produced in step 1340 to produce an Iodide (1352), bimolecular Oxygen (1353), and water (1354).

The elementary steps illustrated in FIG. 1300 and discussed above yield a very neat and clean description for the observations if one ignores the difficulty in obtaining the IOH molecule (1321). Upon close observations holes begin to appear in the mechanisms. The first and most obvious, except in the case of excess concentrations of Iodide ions (1322 and 1342), is the difference in K values for the formation of the IOH molecule (1323) versus the K values for the formation of the Iodide ions (1352) and formation of bimolecular Oxygen (1353). If the Iodide ion (1352) formed in the “disproportionation” reaction (step 1350) must be reused in either step 1320 in place of the consumed Iodide ion (1322) or in step 1340 in place of the consumed Iodide ion (1342), the K value of 1320 at ten to the zero power or even worse the K value of 1340 of ten to the minus 2 power would grind the reaction to a stop. Indeed the K value for step 1350 the “disproportionation” reaction is high being ten to the 8 power, but in order to replace the IOH molecule (1321) consumed in the reaction one must take the Iodide ion (1352) produced by the reaction and plug it back into either step 1320 or step 1340 in which the K values are low. Experimentally, in our lab, we combined 0.294 moles of Hydrogen Peroxide with 0.000602 moles of Iodide in basic solution; a solution in which Hydrogen Peroxide is in very great excess relative to the Iodide. The great excess of Hydrogen Peroxide was completely depleted within thirty minutes in a reaction that started slowly but built into a very rapid reaction. Our experimental observation, therefore, discounts the mechanism. In addition, when one studies the elementary steps suggested by Kessi-Rabbia one notices that the oxidation state of Iodine is never greater than plus 1. Because the other halogens, particularly bromide and chloride, exhibit similar plus 1 oxidation state chemistry, it is reasonable to assume that if the plus 1 oxidation state is the only oxidation state used, the Chloride or Bromide ion would also work in the reaction.

Experimentally, in our lab, the replacement of the Iodide ion with equal quantities and then excess quantities of the Chloride ion or Bromide ion produced no reaction at all over extended periods. We went a step further and conducted two additional experiments.

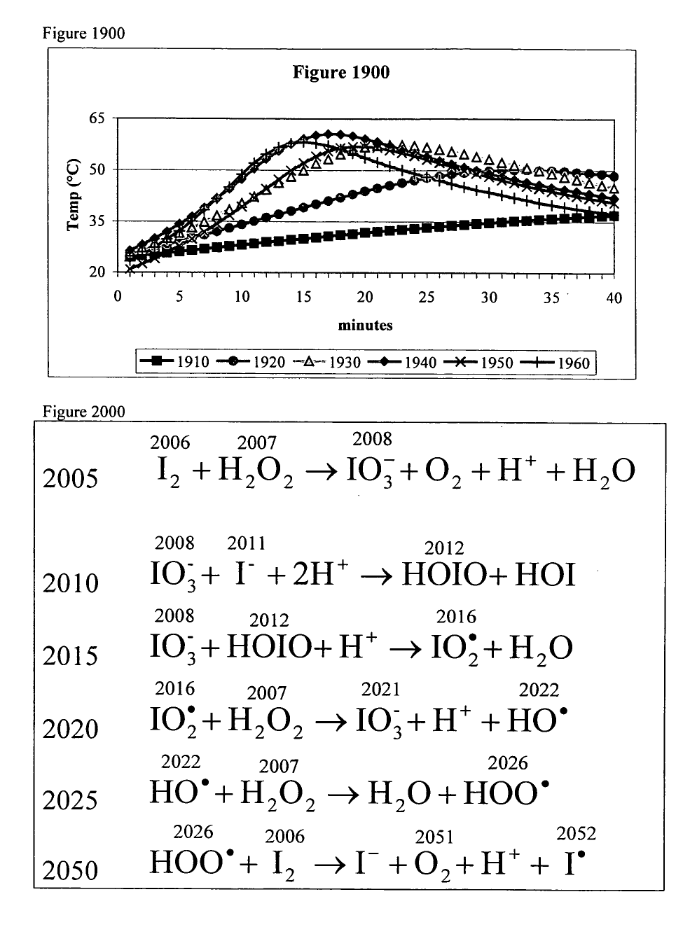

In the first reaction 0.100 grams of KI (0.000602 mole of Iodide) was combined with 0.100 grams of KCl (0.00134 mole of Chloride) and 0.059 grams of KOH in a 10% solution of Hydrogen Peroxide. The pH and Temperature were measured every minute for 40 minutes. A standard solution was then prepared that was identical to the test solution except the KCl was not added. A plot of the data is presented in FIG. 1400. The slope of the reaction temperature (1420 and 1425) and pH change (1410 and 1415) are virtually identical meaning that the addition of excess Chloride (1415 and 1425), relative to Iodide, had no effect on the reaction; chloride neither slowed nor hastened the reaction: it did not play a role. To validate the statement we conducted an experiment in which we increased the concentration of KI over six separate reactions. We first prepared a stock solution which contained a 0.0106 molar concentration of KOH and a 3.235 molar (11%) concentration of Hydrogen Peroxide. This stock solution was then carefully measured out into 100 gram lots for the six separate reactions. To the first reaction 0.050 grams (0.000301 moles or 0.05%) of KI were added. To the second reaction 0.100 grams (0.000602 moles or 0.1%) of KI were added. To the third reaction 0.150 grams (0.000903 moles or 0.15%) KI were added. To the forth reaction 0.200 grams (0.001205 moles or 0.20%) KI were added. To the fifth reaction 0.250 grams (0.001506 moles or 0.25%) KI were added. The sixth reaction contained 80 grams of stock solution to which 0.240 grams (0.00145 moles or 0.30%) KI were added. In each reaction the pH and Temperature values were taken and recorded every minute. The data from these reactions were plotted and are presented in FIG. 1900. The lowest concentration of KI used in this series was 0.05% (1910). The KI concentration was then doubled to 0.10% (1920). The concentration of KI was then increased an additional five tenths of one percent to 0.15% (1930). The concentration of KI was then increased an additional five tenths of one percent to 0.20% (1940). The concentration of KI was then increased an additional five tenths of one percent to 0.25% (1950). The concentration of KI was then increased an additional five tenths of one percent to 0.30% (1960). As the curves (1910, 1920, 1930, 1940, 1950, and 1960) compared it becomes clear that as the concentration of the Iodide ion is increased the reaction rate is substantially increased. From the results of this study one would expect that if Chloride plays any sort of a catalytic role the rate would substantially increase. As it can clearly be seen in FIG. 1400 no such increase exists. Chloride cannot initiate, sustain, or increase the rate of this reaction. This determination is quite important in that Chlorine has every allowable oxidation state that Iodine has: +/−1, +5, +7 and Chlorine even has an additional oxidation state that is not allowed in Iodine: +3. Of course the energy levels of Chlorine's oxidation states are quite a bit higher than Iodine because it is much smaller and much more electronegative. A closer match in size and electronegativity is Bromine.

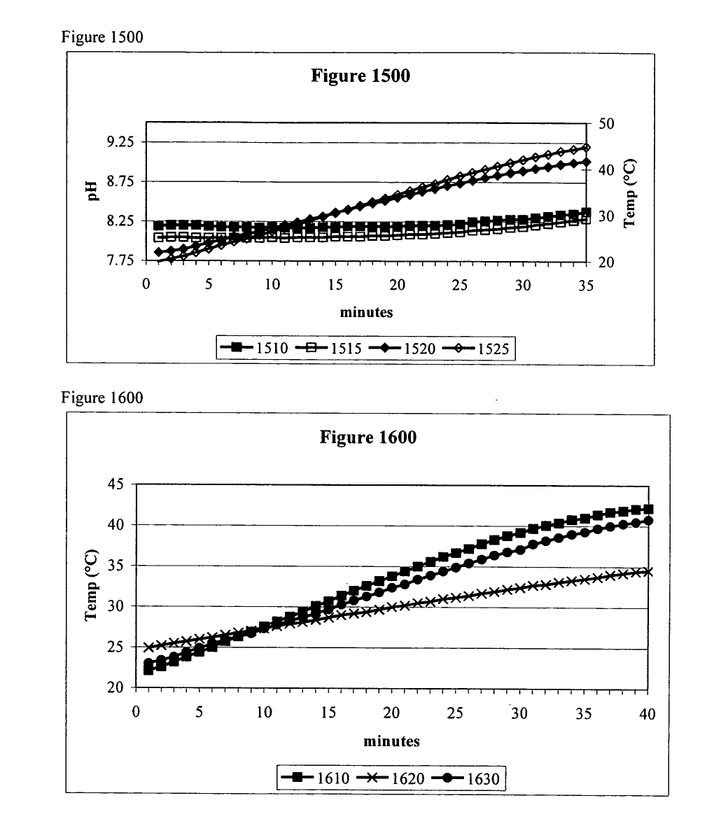

The second experiment was a similar experiment with Bromine. 0.101 grams of KI (0.000608 moles of Iodide) were combined with 0.101 grams of KBr (0.000848 moles of Bromide) and 0.059 grams of KOH in a 10% solution of Hydrogen Peroxide. Again the pH (1510 and 1515) and Temperature (1520 and 1525) were measured every minute for 40 minutes and again, a standard solution identical to the test solution with the notable exception that the Bromide had not been added was prepared and measured in the same manner with the same equipment. The data collected from these reactions is plotted in FIG. 1500. Again the slope of the line, not the ultimate magnitude, determines the rate of the reaction. The slope of the Bromide reaction (1515 and 1525) and the slope of the Iodide reaction (1510 and 1520) are virtually identical; statistically they are identical. To validate this statement we conducted an experiment in which the KI concentration is increased. A discussion of the experiment can be found above in the Chloride ion discussion.

The results of the KI concentration study are plotted in FIG. 1900. As is clearly demonstrated in FIG. 1900 the reaction increased substantially when additional Iodide was added further proving that Bromide, as is true with Chloride, plays no part in this reaction. Bromide cannot initiate, sustain, or increase the rate of this reaction. This finding is important because the size, electronegativity, and orbital configuration of Bromine is a much closer match to Iodine than is Chorine. With respect to the orbital configuration Iodine and Bromine both possess D orbitals whereas Chorine does not. There are two functional differences: The first is that Iodine possess a 4D orbital and Bromine possess a 3D. The second is the allowable oxidation states. Bromine has three allowable oxidation states: +/−1, +5. Iodine has four allowable oxidation states: +/−1, +5, +7. It can be argued that it is the +7 oxidation state and the D4 orbital that makes all the difference and allows the reaction to proceed. However, that argument cannot be applied to the mechanisms described by Kessi-Rabbia. Kessi-Rabbia's mechanism allows for only the +1 oxidation state. In such a +1 oxidation state mechanism, even though chemically different, other halogens, particularly Bromine, would be expected to produce the reaction to some degree. Our research clearly demonstrates that they do not. Kessi-Rabbia mechanism, in short, fails to describe the reaction of Iodide with Peroxide in basic solution. The short fall may be caused by the failure to recognize that free radicals produced by higher oxidation number metastable Iodo-compounds are involved. We wanted to determine the presence of free radicals in the system. We conducted a literature review looking for free radical traps or free radical scavengers. The search yielded the following references:

Hydroxyl radical is the major causative factor in stress-induced gastric ulceration. Das, Dipak; Bandyopadhyay, Debashis; Bhattacharjee, Mrinalini; Banerjee, Ranajit K. Department Physiology, Indian Institute Chemical Biology, Calcutta, India. Free Radical Biology & Medicine (1997), 23(1), 8-18.

Photolysis of chlorpromazine: hydroxyl radical detection using 2-methyl-2-nitrosopropane as a spin trap. Lion, Y.; Decuyper, J.; Van de Vorst, A.; Piette, J. Phys. Inst., Univ. Liege, Liege, Belg. Journal of Photochemistry (1982), 20(2), 169-74.

Mechanistic studies of surface catalyzed H2O2 decomposition and contaminant degradation in the presence of sand. Miller, Christopher M.; Valentine, Richard L. Department of Civil and Environmental Engineering, The University of Iowa, Iowa City, Iowa, USA. Water Research (1999), 33(12), 2805-2816.

Sonolysis of aqueous solutions under argon: dependence of the rate of hydrogen peroxide formation on hydroxyl radical scavenger concentration. Rassokhin, Dmitrii N.; Gokzhaev, Mikhail B.; Bugaenko, Lenar T.; Kovalev, Georgii V. Dep. Chem., M. V. Lomonosov Moscow State Univ., Moscow, Russia. Mendeleev Communications (1994), (1), 25-7.

The above cited references are hereby incorporated by reference.

We determined, from the references, that there were two different free radical scavengers that were readily available and safe to use: Ethanol and Benzoate. Ethanol and Benzoate are effective Hydroxyl Radical (HO.) scavengers.

Two series of experiments were conducted, one in an acidic medium and one in a basic medium.

In the acidic medium, three solutions were prepared containing a final concentration of 10% Hydrogen Peroxide. To the first 100 gram, standard solution, 0.102 grams of KI (0.000614 mole of Iodide) was added and the pH and Temperature were measured and recorded every minute. The second solution contained 97.5 grams of aqueous Hydrogen Peroxide to which 2.500 grams of Sodium Benzoate was added. 0.102 grams of KI (0.000614 mole of Iodide) was added to the solution and the pH and Temperature were measured and recorded every minute. The third 100 gram solution was prepared containing 50% Ethanol. 0.102 grams of KI (0.000614 mole of Iodide) was added to the solution and the pH and Temperature were measured and recorded every minute.

The data were collected, plotted and are presented in FIG. 1600. The slope of the Standard reaction (1610), in which no Benzoate or Ethanol was added, is approximately 0.6-0.7 degrees per minute. The slope of the Benzoate reaction (1620) is approximately 0.4-0.5 degrees per minute. The slope of the Ethanol reaction (1630) is approximately 0.2-0.3 degrees per minute.

In the basic medium, three solutions were prepared containing a final concentration of 10% Hydrogen Peroxide. To the first 100 gram, standard solution, 0.060 grams of KOH (0.00107 mole) and 0.150 grams of KI (0.000904 mole of Iodide) were added and the pH and Temperature were measured and recorded every minute. The second solution contained 97.5 grams of aqueous Hydrogen Peroxide to which 2.500 grams of Sodium Benzoate was added. 0.060 grams of KOH (0.00107 mole) and 0.150 grams of KI (0.000904 mole of Iodide) were added to the solution and the pH and Temperature were measured and recorded every minute. The third 100 gram solution was prepared containing 50% Ethanol. 0.060 grams of KOH (0.00107 mole) and 0.150 grams of KI (0.000904 mole of Iodide) were added to the solution and the pH and Temperature were measured and recorded every minute.

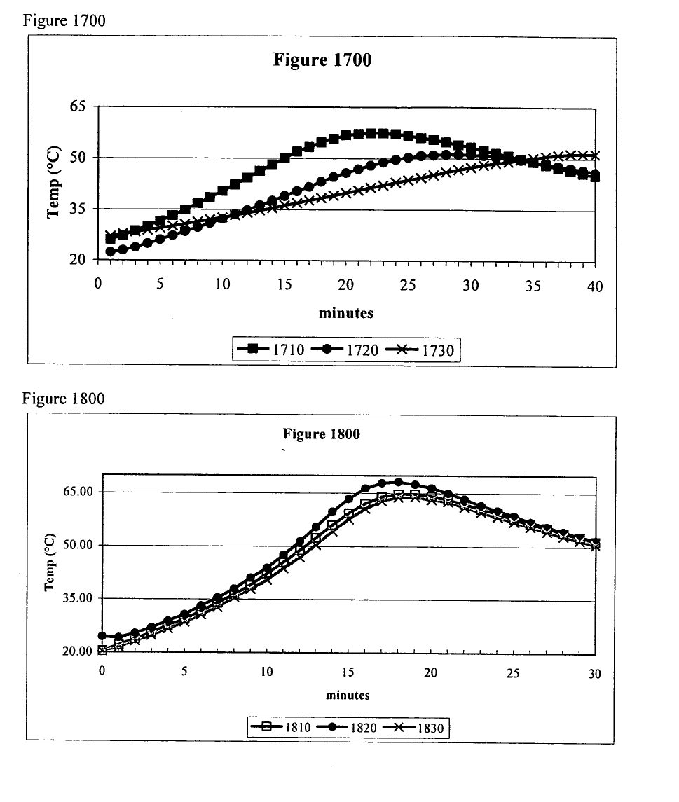

The data were collected, plotted and are presented in FIG. 1700. The slope of the Standard reaction (1710), in which no Benzoate or Ethanol was added, rose sharply and attained a slope in excess of 2.0 degrees in one minute. The slope of the Benzoate reaction (1720) is approximately 1.2-1.4 degrees per minute once a steady state was achieved. The slope of the Ethanol reaction (1730) is approximately 0.6-0.7 degrees per minute.

As one can clearly see in FIGS. 1600 and 1700 the addition of Benzoate or Ethanol slowed the reaction rate in both the acidic medium and the basic medium. Because both Ethanol and Benzoate are known to be and are commonly used as Hydroxyl Radical (HO.) scavengers the obvious conclusion is that Hydroxyl Radicals (HO.) play a role in the reaction whether in acidic medium or basic medium. By way of confirmation that it was the influence of a Hydroxyl Radical scavenger that affected the reaction and not simply the presence of additional ions in the solution, two additional studies were conducted using an inorganic salt, Potassium Nitrate, and an organic salt, Potassium Citrate. A stock 10% Hydrogen Peroxide solution was prepared in a basic medium and 0.2% KI was added. A Standard reaction was conducted and the pH and Temperature was measured and recorded every minute. A second reaction was conducted in which all concentrations remained identical except that the solution contained 2.5% Potassium Citrate. A third reaction was conducted in which all concentrations, again, remained the same as the standard. The only change in the third reaction was the solution contained 2.5% Potassium Nitrate. Again, the Temperature and pH values were collected and recorded every minute.

The data from these reactions were plotted in the graph depicted in FIG. 1800. The slope for the Standard reaction curve (1810), the Potassium Nitrate reaction curve (1820), and the Potassium Citrate containing reaction curve (1830) are virtually identical indicating that these salts had no effect on the reaction.

Based on the results of the reactions which are graphed in FIGS. 1600, 1700, and 1800, the obvious conclusion is that the Iodide catalyzed reaction of Hydrogen Peroxide is not the “disproportionation” of Hydrogen Peroxide in which bimolecular Oxygen is generated directly from the Hydrogen Peroxide molecule described by Kessi-Rabbia, but is a reaction in which, at least in part, free radicals, in particular the Hydroxyl Free Radical (HO.) plays an important role.

The realization that the reaction, in fact, involves free radicals brings us to the remaining two reference citations mentioned earlier, namely: Oscillations in chemical systems. 13. A detailed molecular mechanism for the Bray-Liebhafsky reaction of iodate and hydrogen peroxide. Sharma, Kumud R, et al. and Detailed Calculations Modeling the Oscillatory Bray-Liebhafsky Reaction. Edelson, David, et al. These documents are hereby incorporated by reference.

The Bray-Liebhafsky reaction is conducted in an acidic environment and is very sensitive to pH and may, therefore, be very different than the reaction carried out in a basic environment. Unfortunately, the only substantive work carried out in a basic environment that could be located in the literature search was the work conducted by Kessi-Rabbia, which we just discussed and is based on the incorrect assumption that free radicals are not involved in the reaction. The two references that we are about to discuss both assume and demonstrate free radical involvement and, therefore, may be useful in developing a mechanism that is satisfied in a basic environment. We will begin with the more detailed and earlier of the two first: Oscillations in chemical systems. 13. A detailed molecular mechanism for the Bray-Liebhafsky reaction of iodate and hydrogen peroxide. Sharma, Kumud R, et al.

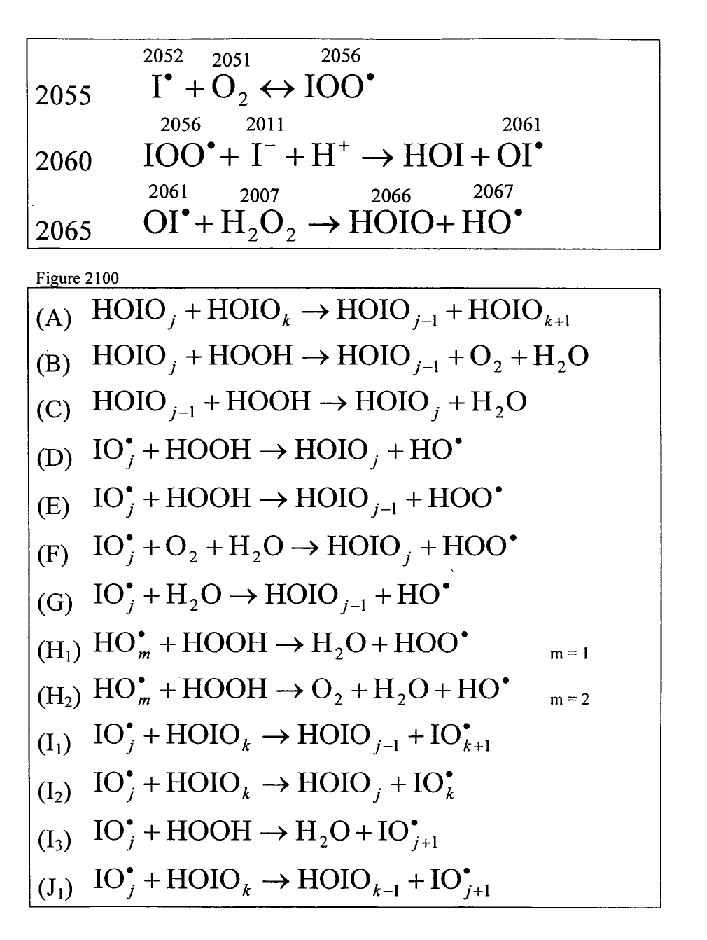

Sharma proposes, in part and summary, the reaction sequence illustrated in FIG. 2000. Sharma begins the sequence (2005) with the reaction of diatomic, elemental Iodine. (It is well known and established that Iodide anions are reduced in acidic medium by Hydrogen Peroxide and that the diatomic, elemental Iodide would reside, at large, in such a solution.) The diatomic, elemental Iodide (2006) reacts with Hydrogen Peroxide (2007) to form the lodate anion (2008). In the next sequence (2010) the lodate anion (2008) reacts with the Iodide anion (2011) to produce Iodous Acid (2012). In the next sequence (2015) more lodate anion (2008) reacts with the freshly created Iodous acid (2012) to yield a Di-oxygen Iodic free radical (2016). In the next sequence (2020) the freshly created Di-oxygen Iodic free radical (2016) reacts with Hydrogen Peroxide (2007) to yield another Iodate anion (2021) which can be used in sequence 2010 to continue the cycle to this point and a Hydroxyl Free Radical (2022). The next sequence (2025) involves the reaction of the freshly created Hydroxyl Free Radical (2022) with Hydrogen Peroxide at large in the solution (2007) to produce a Hydro-Dioxyl Free Radical (2026). The next sequence in the series (2050) reacts the freshly prepared Hydro-Dioxyl Free Radical (2026) with diatomic, elemental Iodide (2006) which resides at large in the solution to yield Diatomic Oxygen (2051) which bubbles out of the solution. The reaction can, by way of the cyclic nature of sequences 2010 through 2020, sustain itself. However, Sharma, establishes a secondary reaction in which the kinetics are more fully explained. Sequence 2050 which yields the Oxygen gas (2051) also yields an Iodine Free Radical (2052). In the next sequence (2055) the Iodine Free Radical (2052) reacts with the freshly created Diatomic Oxygen (2051) in equilibrium to produce a Iodo-dioxy Free Radical (2056). In the next sequence (2060) the freshly created Iodi-dioxy Free Radical (2056) reacts with the Iodide anion (2011) at large in the solution to form an Iodo-oxy Free Radical (2061). In the next sequence (2065) the freshly created Iodo-oxy Free Radical (2061) reacts with Hydrogen Peroxide (2007), which is at large in the solution, to produce Iodous Acid (2066) which can be utilized in sequence 2015 and a Hydroxyl Free Radical (2067) which can be utilized in sequence 2025. Therefore the sequences of 2010 through 2020 are self-sustaining and produces a Hydroxyl Free Radical (2022) and the sequences 2055 through 2065 produce a Hydroxyl Free Radical (2067). Both of these Hydroxyl Free Radicals can then be utilized in sequence 2025 to produce the Hydro-dioxyl Free Radical (2026) that can be utilized in sequence 2050 to produce the Diatomic Oxygen gas. Furthermore, the reaction product of the Iodine Free Radical (2052) in the termination sequence 2050 makes the termination sequence a beginning sequence for the self sustaining cycle of sequences 2050 through 2065. An elegant system indeed. Perhaps too elegant as Sharma also submits, early in the reference, a list of “Generalized Possible Reaction Types” for this reaction. That list is presented in FIG. 2100. As one can clearly see FIG. 2100 notes a large number of potential metastable complexes of Iodine and a large number of different free radicals that could possibly play a role. However, kinetically, Sharma make a strong and convincing argument for the condensed mechanism illustrated in FIG. 2000. We will delay discussing potential similarities and impossibilities of this mechanism as it pertains to the basic environment catalytic reaction of interest until we have review a less detailed and more recent work; our third citation: Detailed Calculations Modeling the Oscillatory Bray-Liebhafsky Reaction. Edelson, David, et al.

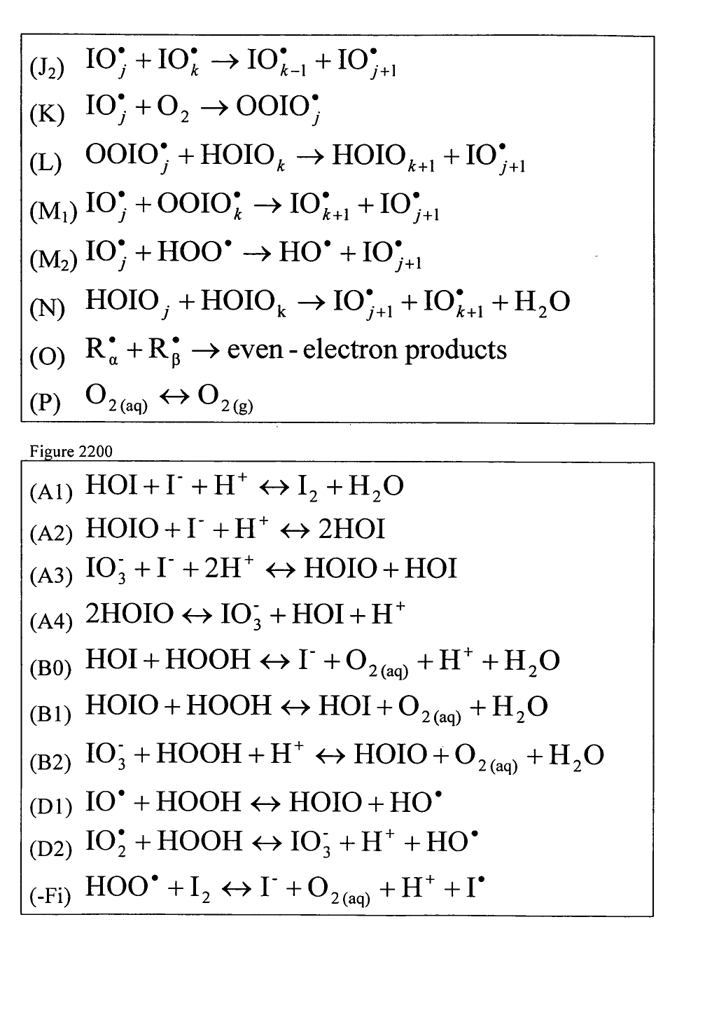

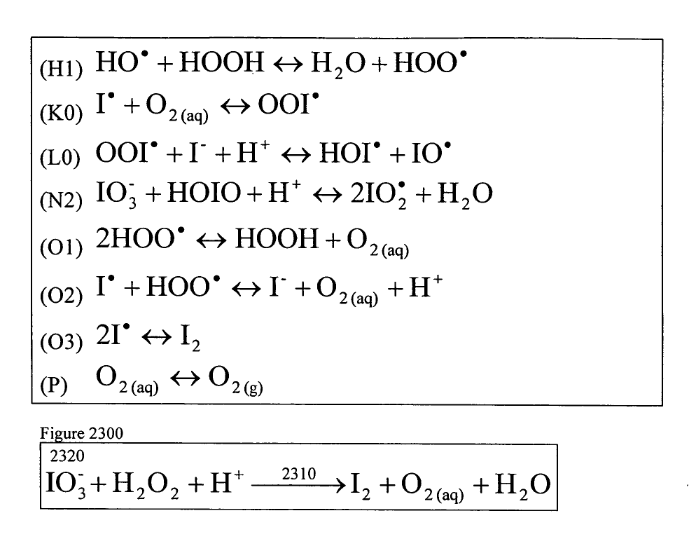

Edelson, whose co-author in this work is the same co-author in Sharma's work, Richard M. Noyes, formulates and validates many of the same reaction sequences as was witnessed in Sharma's work. The possible reaction steps cited in Edelson's work are presented in FIG. 2200. Again, many are identical to sequences in Sharma's work. A very elegant summary reaction of the entire reaction is summarized by Edelson and is presented in FIG. 2300. It is a summarization that explains all observations and can be accepted by all provided that all of the unique, possible, sequences and species illustrated in FIGS. 2100 and 2200 are inserted between the reactants and the products (2310). Indeed, both authors state the complexity of the system in their own unique way. Sharma: “. . . we at first imposed a constraint that no free radical could change its oxidation state by more than 1 equiv in a single elementary process, and we found it was then impossible to accommodate the experimental facts.” And “The development of the mechanism has been a tortuous process. Often the whole effort seemed ready to collapse like a house of cards. A truly intractable experimental fact could still destroy the whole structure. We can only assert that many, many hours of search have been unable to locate that fact!”. Edelson: “If the rate of oxygen escape is assumed proportional to the degree of supersaturation , . . . , the system is too sensitive to changes in concentration of dissolved oxygen. It took us 1 year of frustration before we realized oscillations would only be possible at oxygen concentrations so great that all of the initial hydrogen peroxide would be destroyed long before the necessary conditions were attained.” And “We must admit failure to model closed system behavior well, and at the present time the direction for continued efforts are unclear.”.

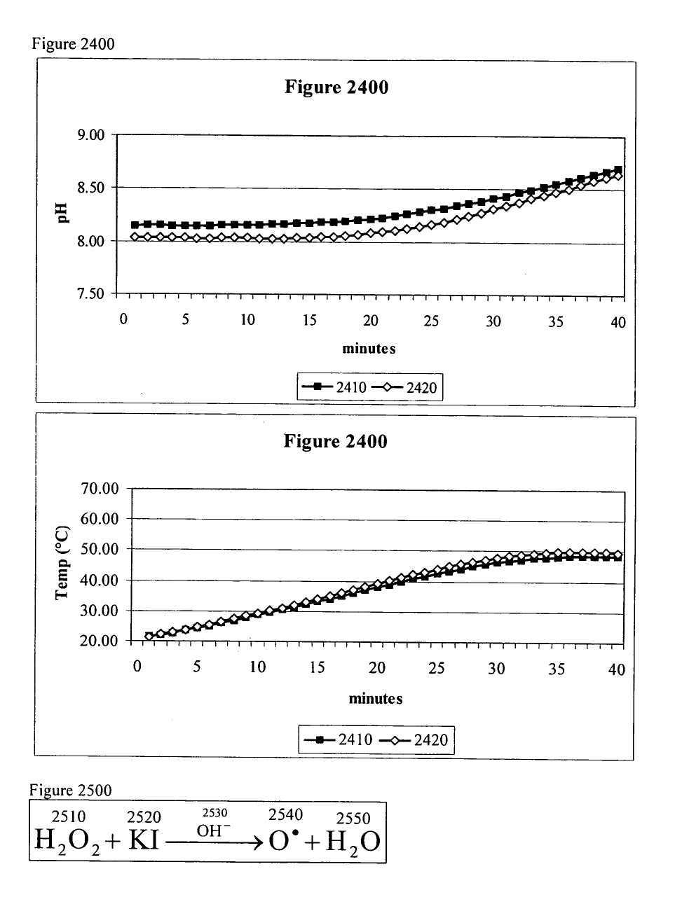

As noted by these researchers the ionic catalyzed “decomposition” of peroxides is a very difficult system to quantify. Far and away the most research has been done in acidic medium and the two citations discussed above are a very good distillation of that work. It was our endeavor to determine which of the sequences discovered and quantified in acidic medium reactions could be applied to the basic medium reactions. To that end we studied the cited work and realized that the lodate ion (FIGS. 2300 (2320) and 2000 (2008 and 2011)) were central to the reaction. To that end we prepared a basic medium reaction and added amounts equal to the Iodide ion, and then amounts in great excess. The addition of the lodate anion did not initiate any reaction even over extended amounts of time. We reasoned, as mentioned by Sharma, that the presence of the Iodide ion may be requisite for the reaction to proceed. To that end we prepared another reaction in which a 10% Hydrogen Peroxide stock solution containing 0.00357 molar concentration of KOH was prepared. In the first Standard reaction 0.120 grams (0.000722 moles) of KI was added to 100 grams of the stock solution. The pH and Temperature values were taken and recorded every minute. In the second reaction 0.120 grams (0.000722 moles) of KI and 0.102 grams (0.000477 moles) of Potassium lodate were added to 100 grams of the stock solution. The pH and Temperature values were taken and recorded every minute. The data was then plotted and those curves are presented in FIG. 2400. As can clearly be seen the rate of the reaction of the Standard solution (2410) and the rate of the reaction of the Potassium lodate solution (2420) are virtually identical: the reaction rate was the same for the Standard solution and the Potassium lodate solution. If the lodate anion plays an essential role in the basic medium as was clearly demonstrated by Sharma and Edelson in the acidic medium, one would expect that the addition of the lodate ion would increase the reaction rate. One would expect to find an increase at least as large as is obtained when one increases the Iodide ion concentration as is illustrated in FIG. 1900. A full discussion of this experiment is present under the Chloride ion discussion above. In brief, the lowest concentration of KI used in this series was 0.05% (1910). The KI concentration was then doubled to 0.10% (1920). The concentration of KI was then increased an additional five tenths of one percent to 0.15% (1930). The concentration of KI was then increased an additional five tenths of one percent to 0.20% (1940). The concentration of KI was then increased an additional five tenths of one percent to 0.25% (1950). The concentration of KI was then increased an additional five tenths of one percent to 0.30% (1960). Clearly as the concentration of Iodide ion increases the rate of the reaction increases. However, refer to FIG. 2400, when the lodate ion is added no such increase or decrease in the reaction rate occurs: the lodate ion plays no role in the basic medium ionic catalytic “decomposition” of peroxides.

There is one major additional difference between the acidic medium reaction and the basic medium reaction: the acidic medium reaction is very pH sensitive, the basic medium reaction is not. As long as the solution is decidedly basic, at a pH of 9 or above, the reaction proceeds well.

One further experimental note; Fluoride being so very electronegative and small was not considered a player in these determinations. Non-the-less an initiation experiment was conducted in which an identical and then very excess amount of Fluoride ion, obtained from Potassium Fluoride, was substituted in the solution for Iodide. After an extended amount of time no reaction was noted. Again, because of the vast chemical differences between Fluorine and Iodine further studies were not conducted. Astatine was not considered because of the availability and health issues involved in dealing with it.

Through the course of our trials we have determined that both the acidic medium and basic medium ionic catalytic “decomposition” of peroxides involve free radicals. In fact, there are a large number of different types of free radicals suggested. It is interesting to note that neither Sharma or Edelson suggest an Oxygen Free Radical (O.) as one of the possible radicals. However, its notable exclusion is easy to explain. Both of these studies are kinetic studies and the Oxygen Free Radical (O.) is virtually impossible to detect, highly reactive, and a very short-lived species making it impossible to study kinetically at this time. Indeed, the presence of the Oxygen Free Radical could be the “truly intractable experimental fact” that Sharma stated “could still destroy the whole structure”. In short, through our own tedious work and the frustrations and sufferings of others, we have learned that the systems are extremely difficult to completely characterize. We believe that Sharma and Edelson propose at least a functional, if not completely accurate, mechanism for the reaction in the acidic medium. We could find no definitive work that as been conducted in the basic medium. We therefore propose our own, very broad, mechanism.

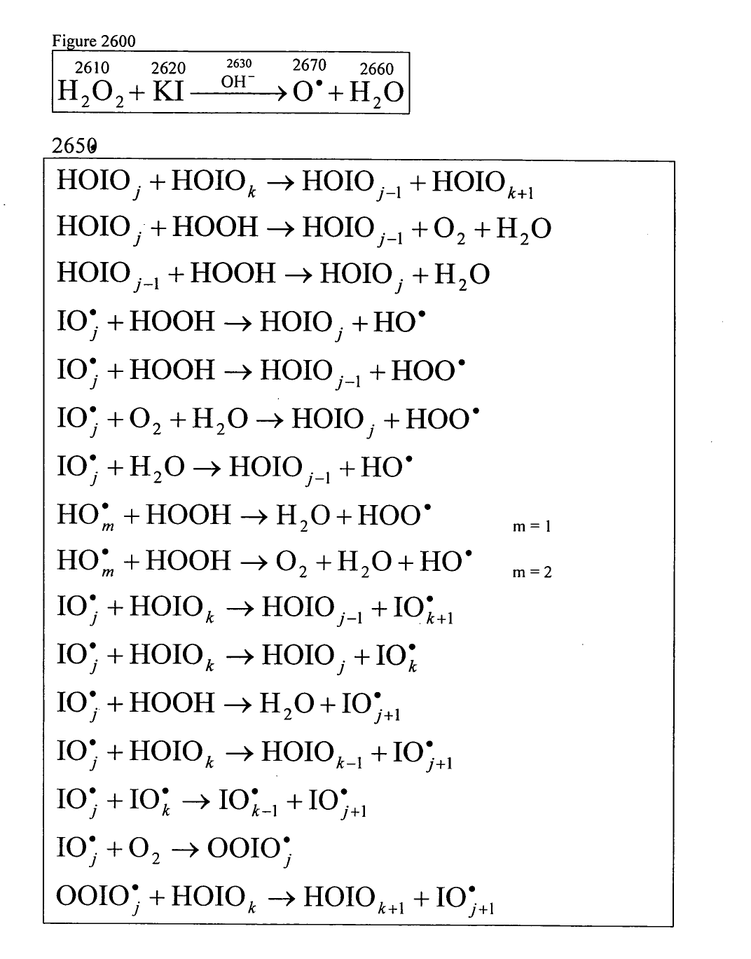

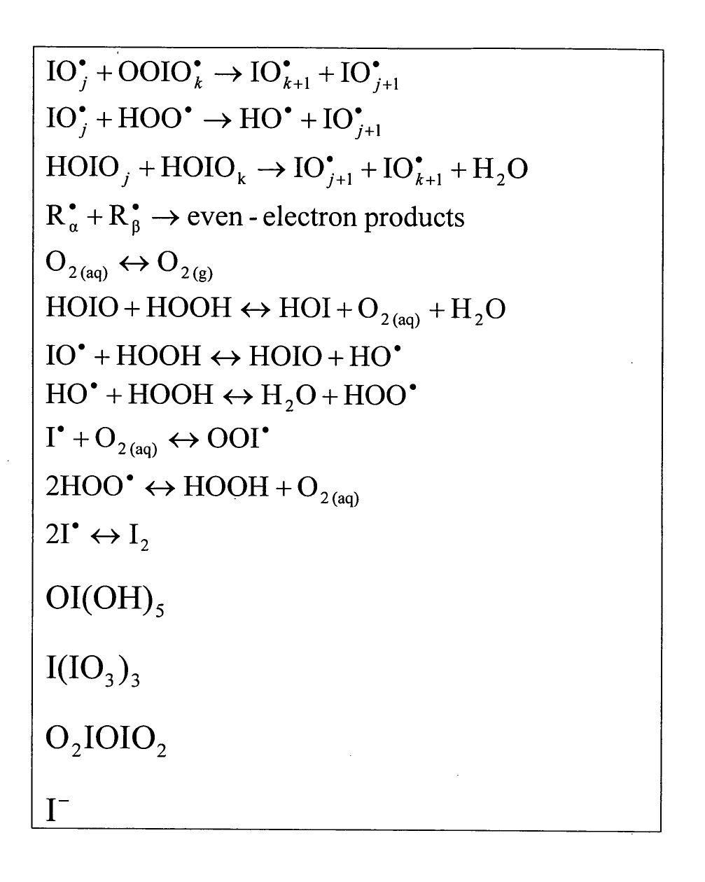

In Edelson form, we advance a simple equation to describe the observed results in FIG. 2500 in which Hydrogen Peroxide (2510) reacts with the Iodide ion produced from Potassium Iodide (2520) in a basic medium (2530) to produce water (2550) and the Oxygen free radical (2540). Also in Edelson form and by way of a more complete explanation we advance the system illustrated in FIG. 2600 in which Hydrogen Peroxide (2610) reacts with the Iodide ion obtained from Potassium Iodide (2620) in a basic medium (2630) to form metastable intermediaries and intermediary reaction as allowed possibilities from our own work and the work of Sharma and Edelson (with the lodate and/or proton requiring species removed) (2650) to produce water (2660) and the Oxygen free radical (2670). Whether the reaction actually produces an Oxygen free radical (O.) or Hydroxyl free radical (HO.) or the Hydro-dioxy free radical (HOO.) or any other possible free radical is very important in terms of quantitative chemistry, however, it is unimportant in whitening and bleaching applications as nearly any free radical, especially any form the Oxy free radical (J-O.), will attack and cleave organic molecules, eliminating their color and increasing their solubility.

For simplicity sake we will dedicate our discussion to the Oxygen free radical (O.) with the understanding that any other free radical allowed in the system could be substituted for the Oxygen free radical (O.). It is further understood and stated that while we have spent a great deal of time discussing the ion catalyzed “decomposition” of peroxides utilizing metal ions, metal oxide ions, and, most predominately, the Iodide ion and Iodide oxide ions there are undoubtedly other ions, whether they be initially cations or anions, that will possess the ability to change certain of their oxidation states to be useful in the catalytic “decomposition” of peroxides and thereby useful to whitening and bleaching applications. For instance, qualitatively, Iron +2, Copper +2, and Lead +2 have proven to be useful catalysts, particularly in a basic environment where the oxides formed appear to change oxidation states and solubilize. In the acidic environment, again qualitatively, the oxides appear to remain as precipitants throughout the reactions. Lead +2 was a particularly potent catalyst with very small quantities depleting the Hydrogen Peroxide in a very short time frame indeed. A gas is generated from this reaction. While not clearly identified yet, the gas' physical properties would lend one to believe that it is Nitrous Oxide, Lead Nitrate being the source for the Lead cation reaction. The point of this dissertation is to demonstrate that we have identified other useful ionic catalysts and surely there are more that we will discover in the future, in short, nothing in this entire presentation should convince the reader to limit the usefulness of any specific ion to the catalytic “decomposition” of peroxides for bleaching and whitening applications.

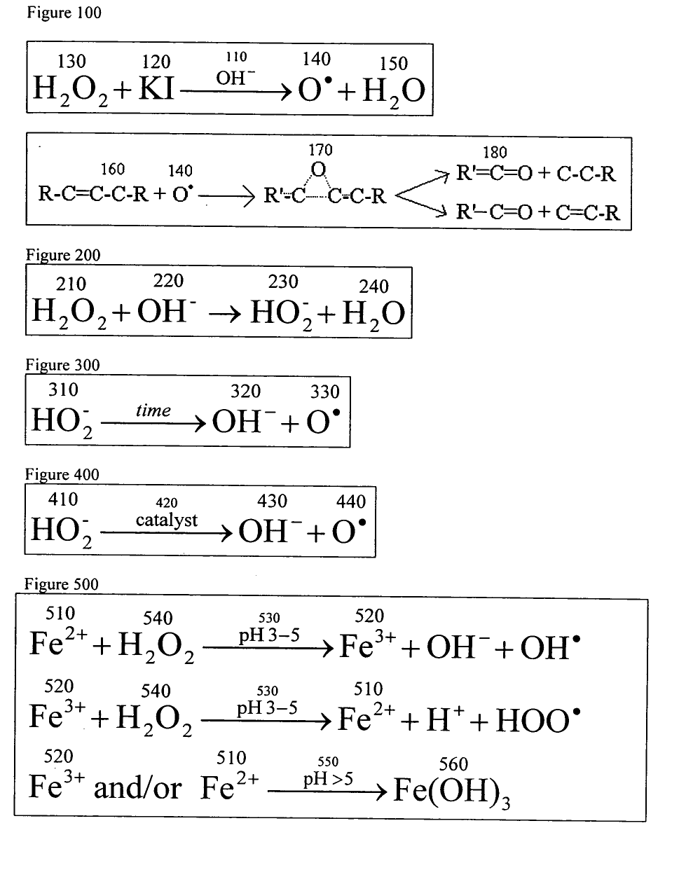

For whitening and bleaching purposes, (refer to FIG. 100) the addition of the Iodide ion by way of Potassium Iodide (120) to a peroxide such as Hydrogen Peroxide (130) in a basic medium (110) yields Free Radical Oxygen (140) and water (150); generating large amounts of heat and depleting the Hydrogen Peroxide in a matter of minutes. The Free Radical Oxygen (140) generated in this reaction can be utilized to oxidize organic molecules that produce offending stains (160) on select items, including teeth. Once the Free Radical Oxygen has oxidized the offending molecule (170) the color is lost and the solubility changes allowing the colorless oxidized fragments (180) of the offending molecule to be washed away in the solvent.

An outstanding application for the ion catalyzed “decomposition” of peroxides in found in tooth whitening or tooth bleaching in which the ionic catalyst is kept separate from the peroxide; a binary system. Such binary systems for tooth whitening or tooth bleaching applications have previously been described in earlier work found in U.S. Pat. No. 6,116,900, “Binary energizer and peroxide delivery system for dental bleaching”, the three pieces of work by Montgomery: “Dental bleaching compositions” (U.S. Pat. Nos.: 5,922,307, 6,322,773, and 6,312,670), the work of Prencipe, et al, “Dual component antiplaque and tooth whitening composition (U.S. Pat. No. 6,110,446), and the work of Allred, “Two-part dental bleaching systems having improved gel stability and methods for bleaching teeth using such systems” (U.S. Pat. No. 6,503,485) all of which are herein incorporated by reference. All of these works describe various systems and methods comprised of a peroxide in a stable gel that is held at an acidic pH which is stored in a separate isolated container or chamber and a second separate isolated container or chamber containing a stable gel with the active ingredients being comprised of elements that provide a basic pH. These systems, thereby, provide a stable storage environment for the two components. When whitening treatment is at hand the two gels are mixed together, the mixture remaining at a basic pH. Of course these systems rely on the painfully slow “decomposition” of peroxides induced by a basic pH. They do not utilize an ionic catalyst to increase or control the “decomposition” rate. However, because they keep the peroxide separate from other ingredients in the system until the moment of use they lend themselves well to the addition of an ionic catalyst.

Recently it has been established that certain potassium salts provide a reduction and even an elimination of sensitivity in hard and soft dental tissues exposed to peroxides during the course of tooth whitening procedures. Illustrations of recent work in this area would include but not be limited to U.S. Pat. No. 6,309,625, Jensen et al, “One-part dental compositions and methods for bleaching and desensitizing teeth” which is incorporated herein by reference and U.S. Pat. No. 6,458,340, Ibsen et al, “Desensitizing bleaching gel” which is incorporated herein by reference. Furthermore, definitive work in the area was done “Clinical evaluation of a combined in-office an at-home applied bleaching agent”, Munoz et al, Loma Linda University School of Dentistry, Center for Dental Research, Loma Linda Calif. which is incorporated herein by reference. All of this work demonstrates that the presence of potassium nitrate and/or potassium citrate, perhaps more specifically the potassium cation, reduces or eliminates sensitivity resulting from bleaching agents. Anionic catalysts and hydronium producing compounds may be obtained in the form of potassium salts thus providing a source of potassium ions and perhaps the additional benefit of desensitization.

The component which does not contain the peroxide can also contain flavorings, sweeteners, additional desensitizers such as the fluoride ion, citrate ion, and/or nitrate ion. The fluoride ion could be introduced to the system by way of its potassium salt thereby increasing the potassium ion concentration and providing perhaps additional value to the system. Additionally, this component could contain elements known to enhance the systems ability to incorporate light into the procedure such as carotene containing dyes and inert colored glass beads; elements which absorb light and convert it to heat. Although, such systems work alone the addition of ultrasonic energy to this system could actually be used to control the reactions, particularly in the presence of thick gels where mobility of the various species is reduced.

The component which contains the peroxide should be limited to compounds required to produce the gel and possibly ion scavengers to provide longer shelf life. Generally ion scavengers chelate to trace metal contaminates in the solution/gel thereby preventing the peroxide from reacting with the trace contaminates and extending the shelf life of the product. Examples of ion scavengers include but are not limited to: Citric acid, alkali metal and alkaline earth metals of citrate, ethylenediaminetetraacetic acid (EDTA) or diaminocyclohexanetetraacetic acid (CDTA) either in their mono-metal salts with sodium or their di-metal salts with sodium and calcium or even more attractive, their potassium salts. Citric acid is desirable for its ability to provide acidic pH stabilization while also being an ion scavenger, however,, the sour taste associated with citric acid reduces its appeal. Ion scavengers are included in concentration ranges from 0.01 to 10%. The addition of other compounds run the risk of reaction with the peroxide.

An additional feature that is supplied first by way of the rapid pH changes involved in the ion catalyzed “decomposition” of peroxide and, second, the highly aggressive oxidation and cleavage of organic molecules by the free radicals produced is that changing colors may be part of the system. For instance if an indicator such as thymolphthalien which is blue in the very basic range and clear in the near neutral range was combined with a dye that is readily attacked and destroyed by the free radicals such as betacarotene, FD&C Red 40, or Amaranth in the non-peroxide compartment, when the peroxide compartment and the non-peroxide compartment contents are mixed the color would immediately change from indigo blue to red. Over a time frame determined by the concentration of dye present, the red color would fade leaving a clear-colorless gel. If allowed to stand long enough the system, as it rebounds to the basic side, would turn a light blue color. The color could be used to demonstrate the system is active as the indigo blue turns to red. The red color could serve two purposes; first and indication that the peroxide is exhausted as the red color fades and second to absorb the blue colored light produced by dental curing lights and lasers if one desires to add such devices to the procedure. Additionally, FD&C Yellow 5 could be added. Yellow 5 is stable in the presence of free radicals generated by this system. The color would then go from indigo blue, to orange, to yellow to green . . . depending on the various concentrations. A number of combinations of pH indicators and dyes are possible with the system and could lend themselves to a variety of uses.

The exact formulations for various mouthwashes has been exhaustively studied and reported. Any of them could be used to formulate a suitable mouthwash with the notable exception of the formulations which contain large (greater than about 5% when both components are mixed) quantities of Ethanol. Ethanol is a known free radical scavenger and retards the reaction as discussed earlier. All chemicals needed to produce a mouthwash are available from the standard chemical sources such as Sigma-Aldrich of Milwaukee, Wis. and Spectrum Chemicals of Gardena, California.

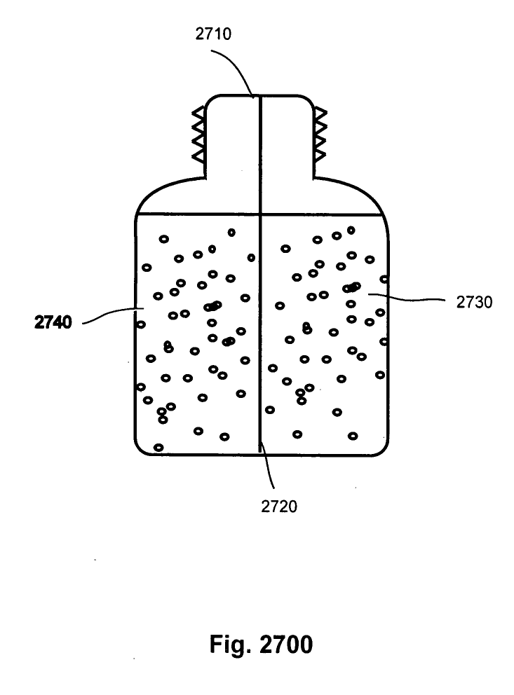

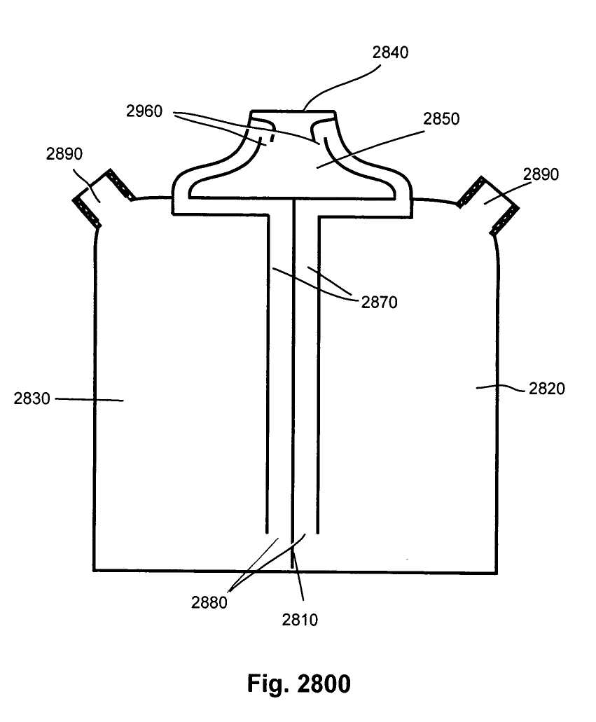

The delivery mechanism and method can be any system that keeps the two components separate until immediately prior to use. They can be as simple as two separate containers in which appropriate amounts of each component, from their respective containers, are poured into a third vessel, such as a drinking glass, and the used according to the instructions. For convenience they can include various two component dispensers. One such simple example is illustrated in FIG. 2700. In such a configuration the peroxide containing component is confined within its own chamber (2710) which is isolated (2720) by a wall from the non-peroxide containing component with its own chamber (2730). In such a configuration the consumer need only pour the desired amount into an appropriate container such as a drinking glass. In such a configuration the consumer would need to take care while pouring to insure that approximately equal portions were dispensed. A more convenient dispensing system is illustrated in FIG. 2800. In such a configuration the peroxide containing component is confined within its own chamber (2810) which is isolated (2820) by a wall from the non-peroxide containing component with its own chamber (2830). A collecting and dispensing cup (2840) is and integral part of the structure. An isolated channel (2850) is provided for each chamber. The isolated channels are open at the bottom of the chamber (2851) and at the top of the dispensing cup (2852). The channels (2850) are open and in full communication with the openings (2851 and 2852) on their respective sides. When force is applied to the sidewalls (2870) the individual components (2810 and 2830) are forced into their respective channel openings (2851), through their respective channels (2850), and out their respective channel openings (2852) located in the collecting and dispensing cup (2840). The components mix together as the collecting and dispensing cup (2840) fills. The consumer may then obtain the mixture directly from the collecting and dispensing cup opening. The filling, pouring and venting ports (2860) provide access in manufacturing to fill the container, may provide ventilation by way of a one way flap or ball valve, and also makes it possible for the consumer to dispense either or both components into other containers. Many other dispensing systems are possible. The above examples are offered for illustrative purposes and are not intended to limit the dispensing systems to the offered examples.

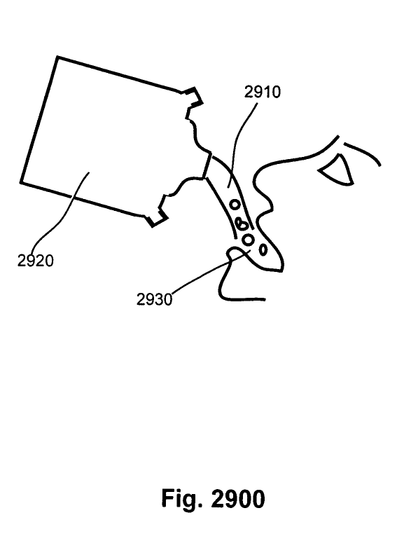

Refer to FIG. 2900. The resultant mixture (2910), dispensed in this illustration by the directly from auto-mixing-dispensing container described above and illustrated in FIG. 2800, is placed in the mouth (2930) and swished across the teeth for the prescribed amount of time that is dictated by the concentration of peroxide and ion catalyst. The used solution is then expelled. The consumer then rinses their mouth with water.

All chemicals in the following example are commercially available from virtually all chemical companies such as Sigma-Aldrich of Milwaukee, Wis. and Spectrum Chemicals of Gardena, California.

### EXAMPLE 1

### Tooth Whitening Mouthwash Containing Low Ethanol Concentrations

| | Energizer Phase | Peroxide Phase |
|---|---|---|
| Ingredient | % by weight | % by weight |
| Menthol* | .043 | 0.0 |
| Thymol* | .064 | 0.0 |
| Methyl Salicylate* | .064 | 0.0 |
| Ethanol | 5.0 | 0.0 |
| Sodium Saccharin | 0.0-5.0 | 0.0 |
| Pluronic .RTM. F127 | 0.3 | 0.0 |
| Magnesium Sulfate | .03 | 0.0 |
| Potassium Hydroxide | .06 | 0.0 |
| Potassium Iodide | .150 | 0.0 |
| Hydrogen Peroxide, 50% aqueous | 0.0 | 6.0-20.0 |
| Ion Scavenger (Di-sodium EDTA) | 0.0 | 0.02 |
| Citric Acid | 0.0 | 0.02 |
| Additional Ingredients (Dyes, | 0.0-10.0 | 0.0 |
| Indicators etc.) | | |
| Deionized or Distilled Water | (QS) | (QS) |

### EXAMPLE 2

### Tooth Whitening Mouthwash Containing Very Low Ethanol Concentrations

| | Energizer Phase | Peroxide Phase |
|---|---|---|
| Ingredient | % by weight | % by weight |
| Menthol* | .085 | 0.0 |
| Thymol* | .128 | 0.0 |
| Methyl Salicylate* | .128 | 0.0 |
| Ethanol | 1.0 | 0.0 |
| Sodium Saccharin | 0.0-5.0 | 0.0 |
| Tauranol .RTM. .sup.2 | 0.6 | 0.0 |
| Magnesium Sulfate | .0.6 | 0.0 |
| Potassium Hydroxide | .06 | 0.0 |
| Potassium Iodide | .150 | 0.0 |
| Hydrogen Peroxide, 50% aqueous | 0.0 | 6.0-20.0 |
| Ion Scavenger (Di-sodium EDTA) | 0.0 | 0.02 |
| Citric Acid | 0.0 | 0.02 |
| Additional Ingredients (Dyes, | 0.0-10.0 | 0.0 |
| Indicators etc.) | | |
| Deionized or Distilled Water | (QS) | (QS) |

### EXAMPLE 3

### Tooth Whitening Mouthwash Containing No Ethanol

| | Energizer Phase | Peroxide Phase |
|---|---|---|
| Ingredient | % by weight | % by weight |
| Peppermint Oil* | 0.0-2.0 | 0.0 |
| Sodium Lauryl Sulphate | .210 | 0.0 |
| Zinc Chloride | .005 | 0.0 |
| Sodium Benzoate | .180 | 0.0 |
| Pluronic .RTM. 105 | 0.5 | 0.0 |
| Glycerine | 7.5 | 0.0 |
| Benzol Alcohol | .04 | 0.0 |
| Polyethylene Glycol | 0.5 | 0.0 |
| Potassium Hydroxide | .06 | 0.0 |
| Potassium Iodide | .150 | 0.0 |
| Hydrogen Peroxide, 50% aqueous | 0.0 | 6.0-20.0 |
| Ion Scavenger (Di-sodium EDTA) | 0.0 | 0.02 |
| Citric Acid | 0.0 | 0.02 |
| Additional Ingredients (Dyes, | 0.0-10.0 | 0.0 |
| Indicators etc.) | | |
| Deionized or Distilled Water | (QS) | (QS) |
| *Some work has been done in the mouthwash arena that demonstrates the combination of Menthol, Thymol, and Methyl Salicylate provides anti-microbial activity which reduces and or prevents plaque. Menthol, Thymol, and Methyl Salicylate also provide flavoring as does Peppermint Oil in Example 3. Any flavoring can be used in the mouthwash by simple replacing the combination of Menthol, Thymol, and Methyl Salicylate in Examples 1 and 2 or the Peppermint Oil | | |
| # in Example 3 with the desired flavoring; .01-10.0% concentrations of flavorings are acceptable. | | |

While compositions and methods have been described and illustrated in conjunction with a number of specific ingredients, materials and configurations herein, those skilled in the art will appreciate that variations and modifications may be made without departing from the principles herein illustrated, described, and claimed. The present invention, as defined by the appended claims, may be embodied in other specific forms without departing from its spirit or essential characteristics. The configurations of snacks described herein are to be considered in all respects as only illustrative, and not restrictive. All changes which come within the meaning and range of equivalency of the claims are to be embraced within their scope.

1. A tooth whitening mouthwash system comprising:
an energizer phase,
said energizer containing an energizer substance that will chemically react with an oxygen-containing medium in a peroxide phase to cause release of oxygen ions therefrom at a rate that is greater than a rate of release of oxygen ions from the oxygen-containing medium absent said energizer substance,
a peroxide phase,
said peroxide phase containing an oxygen-containing medium, and
a storage and mixing vessel,
an energizer chamber within said storage and mixing vessel containing said energizer phase, and
a peroxide chamber within said storage and mixing vessel containing said peroxide phase.

2. A mouthwash as recited in claim 1 wherein said energizer phase includes a basic substance.

3. A mouthwash as recited in claim 1 wherein said energizer phase includes potassium hydroxide.

4. A mouthwash as recited in claim 1 wherein said energizer phase includes potassium iodide.

5. A mouthwash as recited in claim 1 wherein said energizer phase includes both potassium hydroxide and potassium iodide.

6. A mouthwash as recited in claim 1 wherein said energizer phase includes a compound of potassium.

7. A mouthwash as recited in claim 1 wherein said energizer phase includes a compound of iodine.

8. A mouthwash as recited in claim 1 wherein said energizer phase includes a hydroxide and an iodide.

9. A mouthwash as recited in claim 1 wherein said energizer phase includes a thickener.

10. A mouthwash as recited in claim 1 further comprising a thickener selected from the group consisting of polyvinylpyrrolidone, polyvinyl alcohol and glycerin.

11. A mouthwash system comprising:
a vessel,
an energizer chamber within said vessel,
a oxygen radical chamber within said vessel,
wherein contents of said energizer chamber may be mixed with contents of said oxygen radical chamber to form a useful dental bleach,
an energizer phase located within said energizer chamber,
said energizer containing an energizer substance that will chemically react with an oxygen-containing medium in an oxygen-radical phase to cause release of oxygen ions therefrom at a rate that is greater than a rate of release of oxygen ions from the oxygen-containing medium absent said energizer substance,
an oxygen radical phase located within said oxygen radical chamber,
said oxygen radical phase including an oxygen-containing medium.

12. A mouthwash as recited in claim 11 wherein said energizer phase includes a basic substance.

13. A mouthwash as recited in claim 11 wherein said phase includes potassium hydroxide.

14. A mouthwash as recited in claim 11 wherein said energizer phase includes potassium iodide.

15. A mouthwash as recited in claim 11 wherein said energizer phase includes both potassium hydroxide and potassium iodide.

16. A mouthwash as recited in claim 11 wherein said energizer phase includes a compound of potassium.

17. A mouthwash as recited in claim 11 wherein said energizer phase includes a compound of iodine.

18. A mouthwash as recited in claim 11 wherein said energizer phase includes a hydroxide and an iodide.

19. A mouthwash as recited in claim 11 wherein mixture of said energizer phase with said oxygen radical phase results in release of oxygen ions that have a beneficial tooth whitening effect.

20. A mouthwash comprising:
an energizer phase,
said energizer phase containing an element selected from the group consisting of potassium hydroxide and potassium iodide,
an oxygen radical phase,
said oxygen radical phase including oxygen that can be liberated as free radical oxygen.

21. A mouthwash as recited in claim 20 wherein said oxygen radical phase includes hydrogen peroxide.
